(19) United States
(12) Patent Application Publication
(10) Pub. No.: US 2013/0066405 A1
(43) Pub. Date: Mar. 14, 2013

(54) TREATMENT OF SKIN CONDITIONS

(71) Applicant: Gordon Rex Paterson Dougal, Spennymoor, Durham (GB)

(72) Inventor: Gordon Rex Paterson Dougal, Spennymoor, Durham (GB)

(21) Appl. No.: 13/639,597

(22) PCT Filed: Apr. 5, 2011
(86) PCT No.: PCT/GB2011/050677

§ 371 Date: Nov. 27, 2012

(30) Foreign Application Priority Data
Apr. 6, 2010 (GB) 1005662.0

(51) Int. Cl. A61N 5/06

(52) U.S. Cl. CPC A61N 5/0616
USPC 607/94

(57) ABSTRACT

This present invention provides methods of and apparatus for, treating skin conditions such as acne, psoriasis and dermatitis. The methods employ a combination of two anti-phased electromagnetic radiation wavebands having a first peak wavelength of between 1050 to 1085 nm and a second peak wavelength of between 405 to 425 nm, the two specified wavelengths act synergistically to provide improved treatments for chronic and acute skin conditions.

This present invention provides phototherapy methods of, and apparatus for, treating skin conditions, particularly but not exclusively acne, psoriasis and dermatitis. The methods employ a combination of two anti-phased electromagnetic radiation wavebands that act synergistically to provide improved treatments for chronic and acute skin conditions.

### BACKGROUND

Acne vulgaris, or acne, is a skin condition that causes spots and often results in unsightly scar formation which is not only disfiguring but can be psychologically disturbing. Acne can vary from mild to severe and usually affects the skin of the face, back and chest. Acne affects more than four in five teenagers but it is much less common in later life. However, about one in 20 women and one in 100 men aged between 25 to 40 years old continue to be affected by acne, or develop it at this age (late-onset acne). During puberty, both men and women produce more of the male hormone testosterone. This increases the production of sebum in skin, causing it to become greasy and encouraging spots to form. It is thought that this is one of the main reasons most teenagers develop acne during puberty. Acne starts to develop when hair follicles in the skin become blocked with the natural oil (sebum) produced by skin and dead skin cells. Each follicle is connected to a sebaceous gland that lies just underneath the surface of skin, nomally sebum travels up the follicle and out through pores on the surface of the skin. However, if too much sebum is produced, and dead skin cells get trapped in the pores and a ‘spot’ will form, this can either be a whitehead or a blackhead. Sometimes the bacterium Propionibacterium acnes, which normally lives on the surface of the skin, causes inflammation (but not infection) in the hair follicles. If the inflammation develops near the surface of the skin, red or yellow spots (pustules) can form. Deeper inflamed lesions (nodules and cysts) can form if the infection is nearer the hair root. In very severe acne, cysts may join together to form even larger, deeper inflamed lesions (acne conglobata), but this is rare. P. acnes and lesions can also be secondarily infected with Staphyloccocus Aureus.

Dermatologists typically classify types of acne into four grades. Determining the acne grade is done by a simple visual inspection of the skin. Acne lesions are classified by type as grade I (comedone—open or closed), grade II (papule), Grade III (pustule) and grade IV (nodule). Grade I acne is the mildest form of acne. There may be minor pimples but they are small, appear only very occasionally, and in small numbers (one or two). Blackheads and milia will be found, sometimes in great numbers, but there is no inflammation. Grade I acne is commonly seen in early adolescence, especially in the nose and/or forehead but may progress to Grade II if left untreated. Grade II is considered as moderate acne, presenting as greater number of blackheads and milia and more papules and the formation of pustules. In addition slight inflammation of the skin is apparent. Grade II acne may progress to Grade III, especially if pimples are habitually picked at or squeezed. Grade III acne is considered severe. The main difference between Grade II acne and Grade III is the amount of inflammation present. The skin is Obviously reddened and inflamed with Grade III and papules and pustules develop in greater numbers, in addition nodules will be present. Grade III usually involves other body areas, such as the neck, chest, shoulders, and/or upper back, as well as the face. Moreover, the chance of scarring becomes higher as the infection spreads and becomes deeper, left untreated, Grade III acne may progress to Grade IV. Grade IV acne is the most serious form of acne, Grade IV is often referred to as nodulocystic or cystic acne. The skin displays numerous papules, pustules, and nodules, in addition to cysts. There is a pronounced amount of inflammation and breakouts are severe and painful. Acne of this severity usually extends beyond the face, and may affect the entire back, chest, shoulders, and upper arms. The infection is deep and widespread. Nearly all cystic acne sufferers develop scarring. Grade IV acne must be treated by a dermatologist. It tends to be hard to control, and almost always requires powerful systemic medications in addition to topical treatments.

Assessing the grade of acne can help in the choice of the course of treatment that will be most effective. The most commonly prescribed treatments include: benzoyl peroxide; azelaic acid; retinoids such as tretinoin and isotretinoin: antibiotic lotions, such as erythromycin or clindamycin—which can help to control the P. acnes bacteria on skin.

More recently blue light therapy acne treatment has been proposed as an alternative therapy. The light may either be used alone or in conjunction with a topical treatment. The acne blue light treatment has been shown to be effective in treating active acne lesions as well as other sebaceous glands that are not actively producing acne lesions. It has been reported that some of the visible violet light present in sunlight (in the range 415-430 nm) activates a porphyrin (Coproporphyrin III) in Propionibacterium acnes which damages and ultimately kills the bacteria by releasing singlet oxygen. Results have shown that the use of light therapy for three consecutive days has been shown to reduce the bacteria in the pores by 99.9%. Since there are few porphyrins naturally found in the skin, the treatment is believed safe except in patients with porphyria although eye protection is used due to light-sensitive chemicals in the retina. The light is usually created by superluminous LEDs. This form of treatment has been approved by the FDA for some lightwave systems. Overall improvements of on average 76% for 80% of patients occurs over three months; most studies show that it performs better than benzoyl peroxide and the treatment is far better tolerated. However, approximately 10% of users see no improvement.

Other disfiguring and psychologically disturbing skin conditions include psoriasis and contact dermatitis. Contact dermatitis presents as a localized rash or irritation of the skin and is caused by contact with a foreign substance. Inflammation of the affected tissue is present in the epidermis and outer dermis and the itchy rashes may take several days or weeks to fade and heal. Psoriasis is a more serious condition and chronic skin condition where skin cells reproduce about 10× faster and exist in a far higher density than in normal skin, this results in raised, scaly skin patches or plaques. The exact cause of psoriasis is not known however flares can be triggered by infection, stress, changes in temperature or climate, skin injury, some prescribed medicines and alcohol intake. Psoriasis is typically treated by: prescribed topical creams or gels (corticosteroids, vitamin D-, and vitamin A-derivatives); light treatment or phototherapy comprising exposing the affected area to either ultraviolet A (UVA) light alone or with psoralen in order to sensitizes the skin to UV light is prescribed (PUVA); or oral medications such as methotrexate and etretinate, which inhibit skin cell growth and inflammation.

It is also known from the prior art that electromagnetic radiation of a wavelength centered around 1072 nm is effective for the treatment of bacterial and viral infections most notably for the treatment of herpetic infections by reducing the infection time significantly. However, no affect on the treatment of acne, psoriasis or dermatitis has been observed.

A method and apparatus that could effectively treat all grades of acne would offer immediate advantage to acne sufferers.

A method and apparatus that could effectively treat psoriasis and reduce the duration of plaque formation and scarring would offer immediate advantage to sufferers.

A method and apparatus that could reduce the duration of an acne outbreak and concomitantly reduce the amount or level of scarring resulting from the acne outbreak would offer immediate advantage to acne sufferers.

### BRIEF SUMMARY OF THE INVENTION

The present invention resides in the unexpected observation that the particular combination of two specific wavelengths of light potentates the effect in the treatment of certain skin conditions. It has been found that the two narrow bands of specified wavelengths act synergistically to achieve remarkable results in the treatment of acne and psoriasis which is greater than either alone for the same duration of treatment.

According to a first aspect of the invention there is provided a phototherapy comprising a combination of a least two anti-phased electromagnetic radiation wavebands having a first peak wavelength of between 1050 to 1085 nm and a second peak wavelength of between 405 to 425 nm for use in the treatment of a skin condition selected from the group comprising, or consisting of acne, psoriasis and dermatitis.

The at least two electromagnetic radiation wavebands are distinct wavebands insofar as they do not overlap and are of a relatively narrow bandwidth.

Preferably, the waveband of the first peak wavelength is centered around 1072 nm.

Preferably, the waveband of the second peak wavelength is centered around 405-420 nm and more preferably is between 410-415 nm.

Preferably, both the first and second peak wavelengths are pulsed.

Preferably, the first peak wavelength is pulsed from 10 microseconds to 500 microseconds.

Preferably, the repetition rate of the first peak wavelength is between 200 Hz and 900 Hz and more preferably is 600 Hz.

Preferably, the first and second peak wavelengths are not activated simultaneously. In use, the light sources that emit the first and second wavelengths are not operable simultaneously so that neither light source is “on” simultaneously. Accordingly, a patient or user is not subjected to both wavebands at the same time, rather the user is exposed alternately to the first and then the second peak wavelengths in a discrete repetition pattern during a treatment period.

Preferably, the pulse of the second peak wavelength is for the duration that the first peak wavelength is not operating or is “off”.

Preferably, the initial treatment period of the first peak wavelength is between 30 seconds to 3 minutes. The individual being treated is first primed with exposure only to the first peak wavelength, subsequently the treatment is a combination of the first and second peak wavelengths.

Preferably, the treatment period of the second peak wavelength is between 30 seconds to 3 minutes.

Preferably, the total treatment period is for a duration of up to 30 minutes and more preferably is between 2-5 minutes over a particularly affected area of superficial skin. Treatment may be repeated daily for as long as a user requires, typically effective treatment is achieved in a period of several months and up to 6 months.

Preferably, the affected area of skin that receives the light therapy treatment of the present invention is selected from the group comprising face, neck, chest, arms and legs or any other part of the body which is affected by the particular skin condition.

Preferably, the first and/or second peak wavelength electromagnetic radiation is divergent. By divergent it is meant that the electromagnetic radiation emitted from the system of the invention has a divergent half angle of at least 5°. Preferably divergence of the electromagnetic radiation is in the range 15° to 45° half angled divergent.

Preferably, the first and/or second peak wavelength electromagnetic radiation is/are narrow band. By narrow band it is meant that the peak wavelengths are centered around the specified values at preferably +/−10 nm and more preferably +/−5 nm either way from the specified value.

According to a second aspect of the invention there is provided an apparatus for delivering a least two pulsed anti-phased electromagnetic radiation wavebands to an area of superficial mammalian skin, the apparatus comprising:
(i) a movable first light source capable of emitting a first peak wavelength of between 1050 to 1085 nm;
(ii) a movable second light source capable of emitting a second peak wavelength of between 405 to 425 nm; and
(iii) a pre-defined pathway to which the first and second light sources are attached so that in use the said light sources are moved from location to location until an entire treatment area of skin is exposed to the said anti-phased said first and second peak wavelengths.

Reference herein to “anti-phase” is intended to mean that the first and second peak wavelengths are not simultaneously activated so that when one peak wavelength is being emitted the other peak wavelength is not, in this way, when the first peak wavelength is being emitted the second peak wavelength is not. The first and second peak wavelengths are not simultaneously activatable.

Preferably, the pulsed anti-phased electromagnetic radiation wavebands may be of varying pulse width and intensity.

Preferably, the pre-defined pathway is a printed circuit board (pcb) and more preferably is configured so that the movement of the first and second light sources is controlled automatically. Preferably, the apparatus comprises a microprocessor which monitors the intensity of the electromagnetic radiation exposure and ensures the duration of treatment is controlled accurately.

Preferably, the first and second light sources may be housed adjacent one another within a single light emitting module. The first peak wavelength 1072 nm light sources with adjacent second peak wavelength 405 nm-425 nm light sources are arranged in a manner so that the emitted light of the 1072 nm light sources and the 405-425 nm light sources are almost co-incident at the target tissue or affected area of skin.

Preferably, the apparatus comprises a plurality of light emitting modules.

Preferably, the waveband of the first peak wavelength is centered around a 1072 nm emission and preferably the waveband of the second peak wavelength is centered around a 405-420 nm emission and more preferably is between a 410-415 nm emission.

Preferably, the apparatus comprises a series of hinged panels, typically the apparatus comprises a front and two opposing side panels so to create a semicircle so that in use an individual's face is exposed at the front and both sides.

Preferably, each panel has a number of vertical rods or tracks.

Preferably, the light emitting modules are attached to a rod or track so that they can move in a substantially vertical path the length of the rod.

Preferably the system further includes means for fixing the intensity of the radiation within a pre-determined range. The radiation output may be monitored with a visible display indicating correct function of the device both for intensity and wavelength.

Preferably the system further includes means for controlling the duration and intensity of the application of the electromagnetic radiation.

Preferably the light emitting modules comprises a housing or casing within which there is provided a plurality of light emitting means selected from the group comprising LEDs, light emitting polymers, light emitting organic or non-organic polymers and nanocrystals. More preferably the light emitting means are LEDs.

Preferably, the apparatus is either battery operated or mains operated.

Preferably, the apparatus is a hand-held apparatus.

Preferably, the apparatus further includes a visible means to detect if the apparatus is functioning properly or malfunctioning.

According to a third aspect of the invention there is provided a method of alleviating the symptoms of a skin condition selected from the group comprising, or consisting of acne, psoriasis and dermatitis, the method comprising:
(i) exposing an affected area of skin to a first peak wavelength of between 1050 to 1085 nm for a selected period of time; and
(ii) exposing the same affected area of skin to a combination of anti-phased first peak wavelength and a second peak wavelength of between 405 to 425 nm for a further treatment period.

According to a fourth aspect of the invention there is provided a method of reducing the time of an outbreak of a skin condition selected from the group comprising, or consisting of acne, psoriasis and dermatitis, the method comprising:
(i) exposing an affected area of skin to a first peak wavelength of between 1050 to 1085 nm for a selected period of time; and
(ii) exposing the same affected area of skin to a combination of anti-phased first peak wavelength and a second peak wavelength of between 405 to 425 nm for a further treatment period.

According to a fifth aspect of the invention there is provided a method of reducing scarring due to acne, the method comprising:
(i) exposing an affected area of skin to a first peak wavelength of between 1050 to 1085 nm for a selected period of time; and
(ii) exposing the same affected area of skin to a combination of anti-phased first peak wavelength and a second peak wavelength of between 405 to 425 nm for a further treatment period.

It will be appreciated that features ascribed to the first aspect of the invention apply mutatis mutandis to each and every other aspect of the invention.

### BRIEF DESCRIPTION OF THE DRAWINGS

Embodiments of the invention are further described hereinafter with reference to the accompanying drawings, in which:

FIG. 1 shows an apparatus according to the present invention. FIG. 1A shows a side front angled view of the apparatus, FIG. 1B shows a further side angled view of FIG. 1A. FIG. 1C shows a plan view of the apparatus and FIG. 1D shows the operating surface of the apparatus that is in closest proximity to the body surface to an individual that is to receive acne treatment.

FIG. 2 shows a light emitting diode module in accordance with the present invention. FIG. 2A shows a side view of the module, FIG. 2B shows a cut-through plan view of the module and FIG. 2C shows an underneath view of the module.

### DETAILED DESCRIPTION

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The present invention advantageously provides an improved method of treating skin conditions in a cost effective way. Current phototherapy devices treat the entire target area simultaneously which, if the unit cost of the light source is high is prohibitively expensive thus reducing the commercial viability of the product. The apparatus of the present invention provides an improved therapeutic end result but at a reduced cost, the trade off was increased treatment time, which was deemed acceptable by the target consumer group. By means of an example, treating the human face, over one hundred LEDs would be required to treat the entire face simultaneously. The cost of such a device would excessive and would render the product commercially implausible even though the treatment time would be 3-4 minutes. Whilst the apparatus of the present invention concept increases the treatment time to around 20-25 minutes, it does however reduces the cost of the device by 75%. This in turn means that an individual can purchase the device to use in the privacy and convenience of their own home.

The apparatus comprises servo controlled LED array pcbs, which are anchored to each treatment surface via a shaft running the length of the treatment face of the panel from top to bottom. There is at least one pcb but there may be several. The pcb treats a section of the face and then is moved down or up or across the face according to the desired requirement. Once an area is treated the pcb is moved automatically by a motor or electromagnet or any other means to a new location adjacent or distant from the first treatment site. Once the pcb has been moved once or several time the entire area of the face will have been treated.

A man skilled in the art will appreciate that the principle of the apparatus could also be applied to the back and chest and the apparatus modified accordingly.

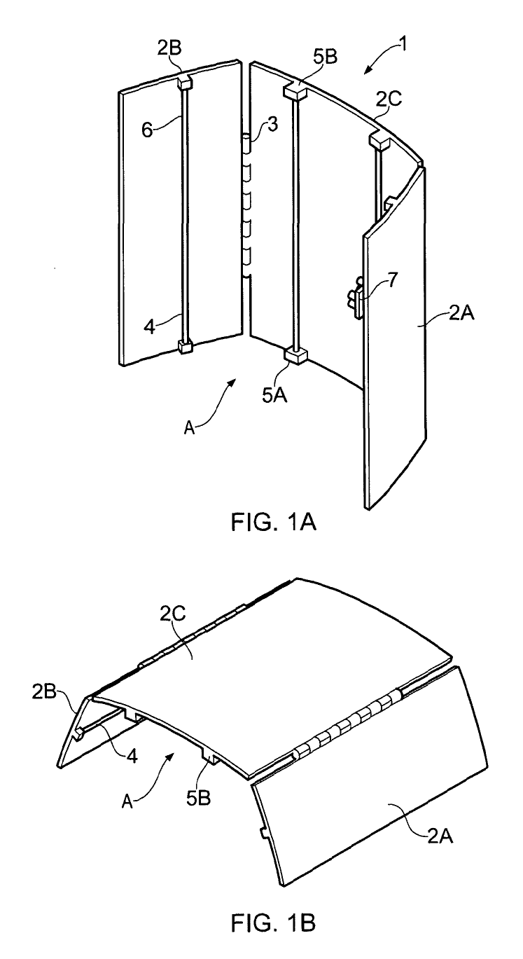

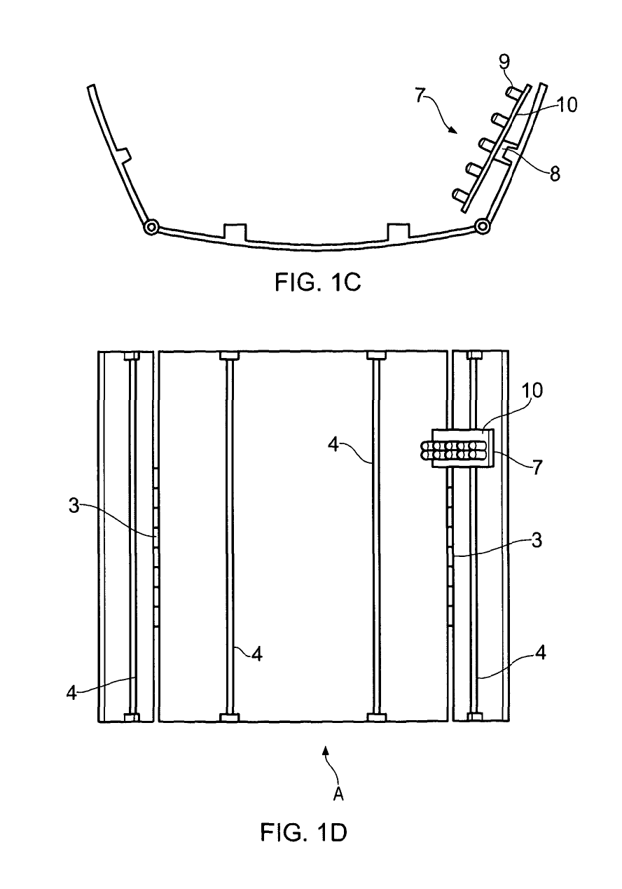

With reference to FIG. 1A there is shown the apparatus (1) of the present invention in a front angled view wherein “A” represents the treatment surface or the surface of the apparatus which in use is in closest proximity to the target area of treatment. The outer construction of the preferred apparatus consists of three or more panels (2A, 2B and 2C) that are hinged (3) between each other. Attached to each of the panels (2A, 2B and 2C) is servo or rod mechanism (4, 6) anchored at each end by attachment means (5A and 5B). The servo or rod mechanism is attached to a printed circuit board (7). FIG. 1B shows an alternative view of FIG. 1A. The light emitting sources (9) are mounted (8) on the printed circuit board (10) (FIG. 1C). Also provided is an electronic motorised or magnetic servo system that enables the printed circuit board (7) to move along the rod mechanism (4) in a predictable controlled manner. The light devices (9) can either be 405 nm to 425 nm light emitting devices or 1072 nm light emitting devices or a combination of each. The light sources are not lasers, but may be LEDs, light emitting polymers, light emitting nanocrystals, pumped light sources which emit light at the desired wavelength. FIG. 1D shows the front view of “A” the treatment surface of the apparatus of the present invention.

The preferred mode of operation of the apparatus is for all the printed circuit boards (7) to be aligned at one end of the panels. In use, the combination treatment of light centered in the range from 405 nm to 425 nm and light centered at 1072 nm is commenced for a period ranging from 2 minutes to 5 minutes. After a period of treatment, the printed circuit boards (7) are moved along the rod mechanism (4, 6) in a controlled predicted manner to enable treatment of another area of skin. The period of treatment is repeated and the printed circuit board moves along the rod mechanism in a predetermined manner until the entire area under the panels (2A, 2B and 2C) have been treated. At the conclusion of the treatments the printed circuit board/light emitting diode array returns to the start position.

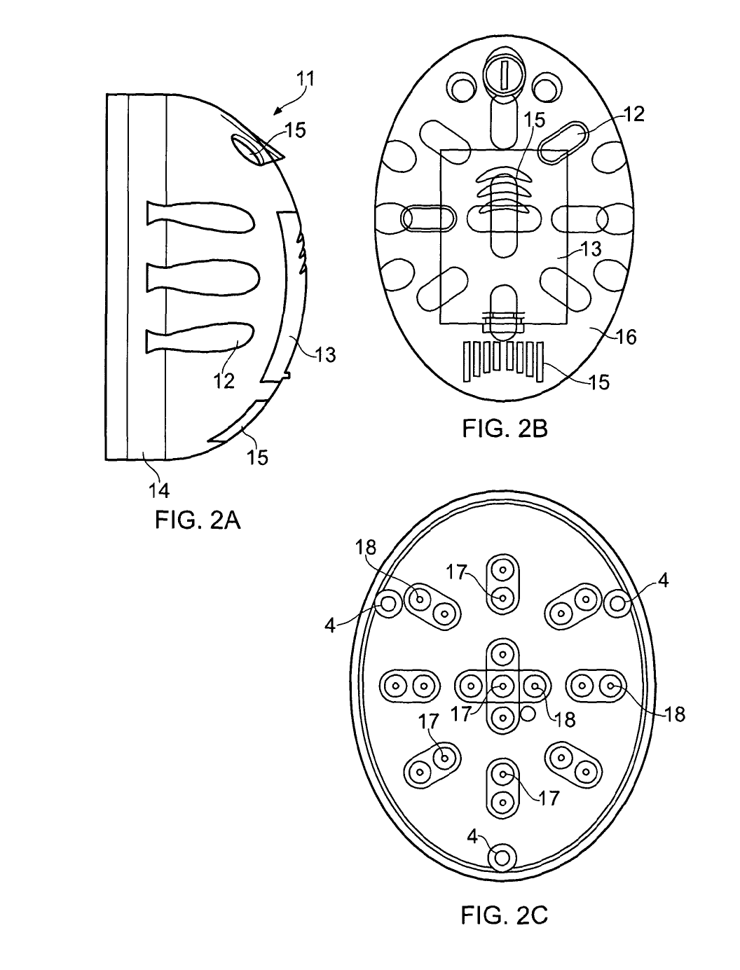

FIG. 2 shows the light emitting modules (11) in greater detail. The module, as seen in a side section (FIG. 2A), comprises a plurality of bulbs (12) anchored in a base (14) the module encapsulates the bulbs (12) and base (14) and immediately above the bulbs is a transparent window (13) through which light can pass to the target area of skin to be treated. Also provided in the module housing are vents (15) so that heat generated from the LED bulbs can be dissipated from the module body (16). FIG. 2B shows a top section of the module (11) and FIG. 2C shows the underside of the module. Light sources that emit 1072 nm (17) are arranged adjacent light sources that emit 405 nm-425 nm (18). The light sources (17) and (18) are arranged in a manner so that the emitted light of the 1072 nm light sources and the 405-425 nm light sources so as to be almost co-incident at the target tissue.

### EXAMPLE 1

A typical treatment protocol comprises exposure of the affected area of skin to a first peak wavelength in the near infer-red region centered around 1072 nm and then to a second peak wavelength in the violet region centered around 415 nm. Both light sources are pulsed so that an individual under treatment only receives one of the peak wavelengths at any given time. The 1072 nm light is pulsed from 10 microseconds to 500 microseconds with a repetition rate in between 200 Hz and 900 Hz, usually at 600 Hz. It is important to note that Importantly both light sources are never “ON” simultaneously. The pulse of the 415 nm light is for the duration that the 1072 nm light is “OFF”. The treatment is commenced using 30 seconds to 3 minutes of 1072 nm light, then the combination of alternated treatment occurs for at least 30 seconds, up to 30 minutes. A normal treatment period is for 3-4 minutes.

### EXAMPLE 2

Clinical data was gathered for 12 patients suffering from varying degrees or severity of acne.

Patients 1-5—These patients had severe scarring due to grade 4/4 pustular acne which had not responded to conventional pharmaceutical therapy. The patients received the combined violet light (405-425) nm and the 1072 nm light treatment for 8 weeks. The redness associated with the active acne was observed to respond within 24 hours and a reduction in the level of skin infection was noted within 5 days. The subcutaneous pustules which are consistent with grade 4 acne were found to be reduced in size and no longer became actively inflamed. After 4 weeks of daily use the pustules largely resolved and did not recur. In addition to the reduction in the pustule and nodule formation, there was a reduction in scar formation associated with active acne and a reduction in the existing scarring of the face, giving an improved appearance of the individual as regards the active acne lesions and the old scars from previous inflammatory acne lesions.

Patients 6-9—These patients had severe nodular acne with severe inflammatory lesions which was not responding to Roaccutane® (isotretinoin), the medication used by dermatologists for the severest forms of acne. These patients had previously tried the 405-425 nm light alone and the 1072 nm light alone with a small but measurable improvement. Subsequently, these patients received the combination of the two wavelengths as hereon before described. These patients reported that the combination of the two wavelengths was substantially superior to either of the sources of light used independently which was a surprising and unexpected therapeutic response. Furthermore, an important finding was the improvement in the inflammatory lesions and the complete absence of new scarring associated with the acne over a period of 11 months. These results show a substantial improvement as compared to the Roaccutanne® which is the global clinical treatment of choice for severe acne.

Patients 10-12—A review of the persistence of the treatment success after completing the course of 420 nm alternating with 1072 nm light treatment. These individuals had grade 3/4 acne affecting their face. After using the combination wavelengths of light as herein before described, 5 times a week for 3 months the patients had a complete resolution of their acne which remained in remission for the ensuing 6 months.

### EXAMPLE 3

In the group of patients 6 to 9 described in Example 1, two of the patients had coexisting facial psoriasis. Following the treatment protocol it was found surprisingly that the psoriasis responded well to the combination of wavelengths, achieving complete resolution of the facial plaque psoriasis in just 14 days. This observation was unexpected as the literature suggests that the only light that is effective in the treatment of psoriasis is UVA and UVB. Moreover, the prior art indicates that 405 nm light is ineffective in the treatment of psoriasis. These results suggest that the combined light of pulsed alternative 1072 nm and 405-425 nm react synergistically to treat psoriasis plaques.

1. A phototherapy comprising a combination of a least two anti-phased electromagnetic radiation wavebands having a first peak wavelength of between 1050 to 1085 nm and a second peak wavelength of between 405 to 425 nm for use in the treatment of a skin condition selected from the group consisting of acne, psoriasis and dermatitis.

2. A phototherapy according to claim 1 wherein the first and/or second peak wavelengths are narrow band wavelengths.

3. A phototherapy according to claim 1 wherein the waveband of the first peak wavelength is centered around 1072 nm.

4. A phototherapy according to claim 1 wherein the waveband of the second peak wavelength is centered around 405-420 nm and more preferably is between 410-415 nm.

5. A phototherapy according to claim 1 wherein both the first and second peak wavelengths are pulsed.

6. A phototherapy according to claim 5 wherein the pulse of the second peak wavelength is for the duration that the first peak wavelength is not operating or is “off”.

7. A phototherapy according to claim 1 wherein the first peak wavelength is pulsed from 10 microseconds to 500 microseconds.

8. A phototherapy according to claim 1 wherein the first peak wavelength has a repetition rate of between 200 Hz and 900 Hz.

9. A phototherapy according to claim 1 wherein the first and second peak wavelengths are not activated simultaneously.

10. A phototherapy according to claim 1 wherein an the initial treatment period of the first peak wavelength is for between 30 seconds to 3 minutes and a subsequent treatment period is with the combined first and second peak wavelengths.

11. A phototherapy according to claim 1 wherein a total treatment period is for a duration of up to 30 minutes.

12. A phototherapy according to claim 1 wherein treatment is repeated daily for a period of weeks to months.

13. A phototherapy according to claim 1 wherein affected area of skin or a target area that receives the light therapy treatment is selected from the group comprising face, neck, chest, arms and legs or any other part of the body which is affected by the particular skin condition.

14. A phototherapy according to claim 1 wherein the first and/or second peak wavelength electromagnetic radiation is divergent, and wherein the divergence of the electromagnetic radiation is in the range 15° to 45° half angled divergent.

15. An apparatus for delivering a least two pulsed anti-phased electromagnetic radiation wavebands to an area of superficial mammalian skin, the apparatus comprising:
(i) a movable first light source configured to emit a first pulsed anti-phased electromagnetic radiation waveband having a first peak wavelength of between 1050 to 1085 nm;
(ii) a movable second light source configured to emit the second pulsed anti-phased electromagnetic radiation waveband having a second peak wavelength of between 405 to 425 nm; and
(iii) a pre-defined pathway to which the first and second light sources are attached so that in use the first and second light sources are moved from location to location until an entire treatment area of skin is exposed to the first and second pulsed anti-phased electromagnetic radiation wavebands at the first and second peak wavelengths.

16. An apparatus according to claim 15 wherein the first and second pulsed anti-phased electromagnetic radiation wavebands are of varying pulse width and intensity.

17. An apparatus according to claim 15 wherein the pre-defined pathway is a printed circuit board (pcb) configured so that the movement of the first and second light sources is controlled automatically.

18. An apparatus according to claim 15 further comprising a microprocessor.

19. An apparatus according to claim 15 wherein the first and second light sources are housed adjacent one another within a single light emitting module.

20. An apparatus according to claim 15 wherein first and second light sources are approximately co-incident at the target tissue or affected area of skin.

21. An apparatus according to claim 15 comprising a plurality of light emitting modules.

22. An apparatus according to claim 15 wherein the first waveband having the first peak wavelength is centered around a 1072 nm emission and the second waveband having the second peak wavelength is centered around a 405-420 nm emission.

23. An apparatus according to claim 15 comprising a series of hinged panels.

24. An apparatus according to claim 23 wherein each panel is associated with one or more vertical rods or tracks.

25. An apparatus according to claim 24 further comprising one or more light emitting modules that house the first and second light sources, wherein light emitting modules are attached to the rod or track so that they for movement in a substantially vertical path the length of the rod.

26. An apparatus according to claim 15 further comprising means for fixing intensity of the electromagnetic radiation within a pre-determined range, wherein the means for fixing intensity is associated with a visible display for indicating correct function of the device both for intensity and wavelength.

27. An apparatus according to claim 15 further comprising means for controlling duration and intensity of application of the electromagnetic radiation.

28. An apparatus according to claim 15 wherein the light source is selected from the group comprising light emitting diodes, light emitting polymers, light emitting organic or non-organic polymers and nanocrystals.

29. An apparatus according to claim 28 wherein the light source are (LEDs).

30. An apparatus according to claim 15 wherein the apparatus is either battery operated or mains operated.

31. An apparatus according to claim 15 wherein the apparatus is a hand-held apparatus.

32. A method of alleviating symptoms of a skin condition selected from the group consisting of acne, psoriasis and dermatitis, the method comprising:
(i) exposing an affected area of skin to a first peak wavelength of between 1050 to 1085 nm for a selected period of time; and
(ii) exposing the same affected area of skin to a combination of anti-phased first peak wavelength and a second peak wavelength of between 405 to 425 nm for a further treatment period to alleviate the symptoms of the skin condition selected from the group consisting of acne, psoriasis and dermatitis.

33. A method of reducing a time of an outbreak of a skin condition selected from the group consisting of acne, psoriasis and dermatitis, the method comprising:
(i) exposing an affected area of skin to a first peak wavelength of between 1050 to 1085 nm for a selected period of time; and
(ii) exposing the same affected area of skin to a combination of anti-phased first peak wavelength and a second peak wavelength of between 405 to 425 nm for a further treatment period to reduce a time of an outbreak of a skin condition selected from the group consisting of acne, psoriasis and dermatitis.

34. A method of reducing scarring due to acne, the method comprising:
(i) exposing an affected area of skin to a first peak wavelength of between 1050 to 1085 nm for a selected period of time; and
(ii) exposing the same affected area of skin to a combination of anti-phased first peak wavelength and a second peak wavelength of between 405 to 425 nm for a further treatment period to reduce scarring due to acne.

35. (canceled)
